(19) United States
(12) Patent Application Publication
(10) Pub. No.: US 2026/0250627 A1
(43) Pub. Date: Aug. 27, 2026

(54) A new bacterial strain Weissella cibaria, a composition comprising it, a pharmaceutical composition for use, a dietary supplement, a probiotic preparation, a bacterial starter culture for making bread, sourdough, bread, a method for the production of bread, a method for microbiological production of dextran, a bacterial preparation and applications using this strain

(71) Applicant: INSTYTUT BIOCHEMII I BIOFIZVKI PAN, Warszawa (PL)

(72) Inventors: Magdalena KOWALCZYK, Warszawa (PL); Tawakalt AYODELE, Warszawa (PL); Jakub BORECZEK, Warszawa (PL); Joanna RADZIWlLL-BIENKOWSKA, Warszawa (PL); Przemyslaw SALANSKI, Debinki (PL); Malgorzata LOBOCKA, Warszawa (PL); Olha KOSTIUCHENKO, Warszawa (PL); Jacek BARDOWSKI, Warszawa (PL)

(21) Appl. No.: 19/160,743

(22) PCT Filed: Feb. 23, 2024
(86) PCT No.: PCT/PL2024/050016

§ 371 Date: Aug. 28, 2025

(30) Foreign Application Priority Data
Feb. 28, 2023 (PL) P.443929

(51) Int. Cl. C12N 1/205; A21D 8/04; A61K 35/00; A61K 35/744; A61P 35/00; C12P 19/08; C12R 1/01

(52) U.S. Cl. CPC C12N 1/205; A21D 8/045; A61K 35/744; A61P 35/00; C12P 19/08; A61K 2035/115; C12R 2001/01

(57) ABSTRACT

An object of the invention is a new bacterial strain Weissella cibaria deposited as KKP 2094p, a composition containing it, a pharmaceutical composition for use as a medicine, a dietary supplement, a probiotic preparation, a bacterial starter culture for making bread, sourdough, bread, a method for the production of bread, a method for the microbiological production of dextran, a bacterial preparation and applications using this strain especially for the prevention and/or treatment, preferably the treatment of intestinal cancer, preferably colorectal cancer.

### TECHNICAL FIELD

The object of the invention is a new strain of lactic acid bacteria Weissella cibaria, deposited on Feb. 6, 2023 (06.02.2023) as KKP 2094p at the IAFB collection (KKP collection), i.e. in the IAFB-SRI Collection of Industrial Microorganisms of prof. Waclaw Dabrowski (IBPRS-PIB Instytut Biotechnologii Przemysłu Rolno-Spożywczego im. prof. Wacława Dąbrowskiego—Państwowy Instytut Badawczy w Warszawie, Polska, IAFB—Institute of Agricultural and Food Biotechnology of prof. Waclaw Dabrowski—State Research Institute in Warsaw, Poland, IAFB-SRI) (IAFB, Culture Collection of Industrial Microorganisms, prof. Waclaw Dabrowski, IAFB-SRI). The object of the invention is also a composition comprising Weissella cibaria KKP 2094p, a pharmaceutical composition for use, a dietary supplement, a probiotic preparation, a bacterial starter culture for making bread (making bakery products), sourdough, bread (bakery), a method for the production of bread (bakery), a method for microbiological production of dextran, a bacterial preparation and applications using this strain particularly for the prevention and/or treatment, preferably the treatment of intestinal cancer, preferably colorectal cancer.

The biomass of the strain Weissella cibaria KKP 2094p is used for biotechnological production of dextran, for making sourdoughs, and for application as a probiotic. The strain was selected for optimal technological effect in biotechnological production of dextran, preferable technological properties in the production of sourdoughs and sourdough bread, and for the preferable health-promoting properties for probiotic applications, and the ability to survive in conditions imitating the digestive tract.

The object of the invention is also a use of the bacteria strain Weissella cibaria KKP 2094p for the production of dextran, for the construction of starter cultures for the production of sourdoughs and sourdough bread, and as a probiotic strain with preferable properties in the skin and in gastrointestinal tract environments. The strain Weissella cibaria KKP 2094p shows high dextran production efficiency, antimicrobial and anticancer activities, high resistance to bile salts and low pH, it can be used for biotechnological production of dextran, as a strain with preferable technological properties for the production of sourdoughs and sourdough bread, and as a probiotic strain.

### STATE OF THE ART

A bacterium of the genus Weissella was discovered for the first time in 1993 in fermented sausages. This genus belongs to the Lactobacilluseae family, which belongs to the well-known group of lactic acid bacteria (LAB) (Collins et al., 1993). Weissella spp. are Gram-positive, catalase-negative, their cocci or rod-shaped cells do not form endospores. Weissella strains and species have long been isolated from various environments and have various applications. Fermented food products such as yogurt, sourdough and kimchi are among the sources from which Weissella bacteria can be isolated (Mun and Chang, 2020; Valerio et al., 2020). Weissella strains play a key role in the shaping of the flavor and texture of foods during fermentation.

Most of the scientific literature on Weissella spp., in particular Weissella confusa and Weissella cibaria, focuses on the production and characterization of exopolysaccharides (EPS) (Hu and Gänzle, 2018).

W. confusa and W. cibaria can produce dextrans with high masses. These polysaccharides increase the elasticity of bread, and their use in gluten-free baking seems promising (Wolter et al., 2014). In cosmetic products, they are used as moisturizing agents and rheology modifiers (Yildiz and Karatas, 2018). Dextran has a soothing effect and it improves skin hydration. It is an emulsion stabilizer. It retains moisture on the skin surface and prevents cosmetics from drying out.

EPS produced by LAB have gained popularity over the years due to their possible functional and technological features (Lynch et al., 2018). It was shown that Weissella and Leuconostoc produce significant amounts of EPS among LAB (Kavitake et al., 2016). In particular, the genus Weissella is responsible for significant dextran production (Kim et al., 2008). Weissella hellenica, W. confusa and W. cibaria are EPS producers of the genus Weissella, which produces many EPS having different structures and chemical compositions. Bacteria of the genus Weissella use the extracellular polysaccharide synthesis pathway to produce glucan, dextran, levan, galactan and fructan, which are homo or heteropolysaccharides (Zhou et al., 2018). EPS production is influenced by many sugars, which include monosaccharides such as glucose, mannose, galactose, fructose, and oligosaccharides: lactose; saccharose, raffinose. The unique technological features of EPS of LAB make them occupy an important position in the food industry as effective emulsifiers, thickeners, stabilizers or texture improvers (Han et al., 2016; Zhang et al., 2018). Buksa et al. recently demonstrated that 1.5% dextran reduces the formation of resistant starch in starch pastes during storage. These works provide new information on hindering the formation of resistant starch by the use of EPS, which can be efficiently produced in sourdough, thereby improving the properties of sourdough bread (Buksa et al. 2021).

EPS have also amazing biological properties due to prebiotic, antioxidant, antiviral, anticancer and immunomodulatory activity (Badel et al., 2011; Saadat et al., 2019; Xu et al., 2017). To be classified as a prebiotic, non-digestible substances, including i.a. EPS, must stimulate the growth of probiotic bacteria more than that of the bacteria present in the intestine. It was shown that dextran produced by W. cibaria stimulates the growth of L. plantarum, L. acidophilus, B. animalis, B. bifidum and B. infantis more than E. coli and E. aerogenes (Baruah et al., 2016). This activity confirmed the classification of dextran as a prebiotic compound.

Literature data on various strains of lactic acid bacteria indicate that the amount of EPS produced is usually higher than 1 g/l (Torino et al., 2015; Vasanthakumari et al., 2015), and in the case of several strains it reaches a value of approximately 10 g/l (Zeidan et al., 2017). The highest dextran production efficiency was recorded as 24.8 g/l for the strain W. cibaria 27 isolated from kimchi (Kavitake et al., 2020). A recent analysis of EPS produced by selected four strains of Weissella confusa/cibaria showed differences in EPS production efficiency ranging from 3.2 g/l to 47.1 g/l and allowed the type of EPS produced to be determined as dextran (Buksa et al. 2021).

The availability of new LAB strains with pro-technological properties is of great importance for the industry. Therefore, researchers are focusing on searching for wild type strains from natural sources to design new, industrially interesting starters, adjunct or bio-protective cultures, as well as probiotics. Recent advances in microbiome research have increased interest and paved the way for studying the mechanisms of action of probiotics. (Tao et al., 2017). In addition to bacteria from the former Lactobacillus genus, there are many other non-pathogenic LAB strains with the renowned GRAS (Generally Recognized as Safe) status provided by the FDA in the USA or QPS (Qualified Presumption of Safety) status in Europe, which may be taken into account when selecting new probiotic strains. There is increasing number of evidences that selected strains of Weissella sp., which play an important role in many industrial and food fermentation processes, have a probiotic activity. It is known that bacteria of the genus Weissella inhabit the intestines of vertebrates, including humans (Lee et al., 2012). To date, it has been found that these bacteria affect intestinal permeability (Prado et al., 2020), reduce depression (Sandes et al., 2020), regenerate intestinal epithelial cells (Prado et al., 2020), affect the metabolism (Elshaghabee et al., 2020) and can kill harmful bacteria (Dey and Kang, 2020). Moreover, it is also known that some strains of Weissella sp. have a beneficial effect on human health, i.a. the maintaining a healthy oral cavity (Kang et al. 2019), treatment of atopic dermatitis and some cancers (Teixeira et al., 2021), they have antitoxic, anticancer and immunomodulatory activity (Yu et al., 2019a; 2019b; Le et al. 2020), but up to date, it is a LAB group that has not been included in the list of species with QPS status published by EFSA (European Food Safety Authority). The use of these bacteria as probiotics requires safety analyses (EFSA, 2018). This is the reason for the limited use of this group of LAB as commercial starter cultures in the food industry or as probiotic strains.

In order for microorganisms to be used as probiotics, they must be non-pathogenic and non-virulent and cannot carry antibiotic resistance genes, in particular on mobile genetic elements. In addition, these microorganisms should have functional features that have a beneficial effect on the host's health. The ability to live and survive in the conditions of the digestive tract, especially at high concentrations of bile salts and low pH, as well as adhesion to mucus, extracellular matrix or intestinal epithelial cells are the most important features of probiotics supporting their action (Lee et al., 2015). W. cibaria can produce several substances having antimicrobial activity. It has been documented that the probiotic strain for use in the oral cavity (W. cibaria CMU) showed antibacterial activity against pathogenic bacteria Porphyromonas gingivalis, Prevotella intermedia, and Fusobacterium nucleatum, related to the ability to produce organic acids, H₂O₂, oleic acids and proteins such as N-acethylmuraminase (Lim et al., 2018). In other studies, the same strain (W. cibaria CMU), as well as the strain W. cibaria CMS1, showed antimicrobial activity and inhibited biofilm formation by strains of pathogenic bacteria causing upper respiratory tract infections such as: Streptococcus pyogenes, Staphylococcus aureus, Streptococcus pneumoniae, Moraxella catarrhalis (Yeu et al., 2021). Research by Patrone et al. (2021) showed that culture supernatants of W. cibaria SP7 and SP19 strains inhibited the growth of Escherichia coli ATCC 25,922 and Salmonella enterica UC3605, what was associated with low pH. W. cibaria strains not only exhibit antagonistic effects against pathogenic bacteria, but may also have an increased ability to effectively reduce the level of fungal infections (Ndagano et al., 2011; Lan et al., 2012).

Currently, the problem of increasing antibiotic resistance among many bacterial pathogens requires new strategies for detecting and developing antibiotics or other antimicrobial agents as an alternative to conventional antibiotics. S. aureus, resistant to many antibiotics, is one of the most common causes of severe hospital infections, and the gastrointestinal tract is an important source of its transmission. (Boyce et al., 2007; Onanuga and Temedie, 2011). At the same time, staphylococci have been recognized as the pathogens responsible for antibiotic-related diarrhea in humans (Boyce and Havill, 2005). Human colonization by facultative bacterial pathogens such as S. aureus is a major risk factor for invasive infections. In recent years, the number of infections caused by both S. aureus and Staphylococcus epidermidis and other coagulase-negative staphylococci has been increasing. These bacteria may cause inflammation of joints, tendons, endocardium, bones, lungs, urinary system, joint prostheses, heart valves, catheters, food poisoning and skin infections such as impetigo, folliculitis, boils, staphylococcal exfoliative arthritis (SSSS syndrome—staphylococcal scalded skin syndrome), but they can also secondarily infect skin lesions, e.g. ulcers. (Sokołowska-Wojdyło, 2021). In this context, it is highly desirable, i.a., to develop preparations based on natural ingredients that will maintain [the] hygiene of the skin by protecting it against colonization by pathogenic bacteria without disturbing the composition of the natural, commensal microbiota and oral probiotics that limit the development of staphylococci in the human digestive tract, and thus minimize the risk of transmitting multi-antibiotic-resistant pathogens from one of their main reservoirs in the environment.

From the publication of the patent description PL238153B1, a starter culture containing the strains of Lactobacillus plantarum B/00117, Lactobacillus plantarum B/00118 and Lactobacillus brevis is known, described as Set 11, used to produce sourdoughs from whole meal and rye flour and to bake bread using thereof. Similarly, the second known starter culture for acidifying of rye flour is the bacterial-yeast culture LV2 by the Lesaffre company, comprising Saccharomyces chevalieri, Lactobacillus brevis-data from the company Lesaffre Bio-Corporation S.A., Łódź.

### DISCLOSURE OF THE INVENTION

In view of the described state of the art, the object of the present invention is to overcome the indicated disadvantages and provide a selected new strain of lactic acid bacteria of the genus Weissella sp. having a high efficiency of EPS production and having preferable technological properties for the production of sourdoughs and bread, at the same time having additional preferable health-promoting properties for probiotic and anticancer applications, the ability to survive in conditions imitating the digestive tract and confirmed safety of use (acceptable level of antibiotic resistance, no antibiotic resistance genes, no virulence genes).

The invention relates to a new bacterial strain Weissella cibaria, deposited in the IAFB collection (KKP collection) (IAFB Collection of Industrial Microorganisms of prof. Waclaw Dabrowski, State Research Institute in Warsaw, Poland) under the deposit no. KKP 2094p.

The invention also relates to a composition comprising the new bacterial strain Weissella cibaria, deposited in the IAFB collection (KKP collection) (IAFB Collection of Industrial Microorganisms of prof. Waclaw Dabrowski, State Research Institute in Warsaw, Poland) under the deposit no. KKP 2094p.

The composition is preferably a pharmaceutical composition further comprising a pharmaceutically acceptable carrier, wherein the composition is preferably in the form of a liquid, solid, powder, tablet, capsule intended for oral administration.

The invention further relates to a pharmaceutical composition for use as a medicament comprising the bacterial strain Weissella cibaria of the invention and/or the composition of the invention and a pharmaceutically acceptable carrier.

Preferably, the pharmaceutical composition for use as the medicament is for use in the prevention and/or treatment of intestinal cancer, preferably in the prevention and/or treatment of colorectal cancer, most preferably intestinal/colorectal adenocarcinoma.

The invention also relates to a dietary supplement comprising the bacterial strain Weissella cibaria of the invention and/or the composition of the invention, wherein the dietary supplement is preferably in the form of a liquid, solid, powder, tablet, capsule intended for oral administration.

The invention also relates to a probiotic preparation comprising the bacterial strain Weissella cibaria of the invention and/or the composition of the invention, the probiotic preparation preferably further comprises prebiotic substances, preferably selected from oligosaccharides, polysaccharides, fructo-oligosaccharides, lactulose, inulin, resistant starch, cellulose, hemicellulose and pectins, the preparation preferably further comprises postbiotics, preferably the postbiotics are selected from short-chain fatty acids, enzymes, lipopolysaccharides, teichoic acids, vitamins, butyric acid, acetate, propionate, muramyl dipeptide, indole, teichoic acid, lactocepins.

The probiotic preparation is preferably in the form of a liquid, solid, powder, tablet or capsule intended for oral administration.

The invention also relates to a bacterial starter culture for making bread, which comprises the strain Weissella cibaria of the invention.

The invention also relates to a sourdough for making bread which contains the bacterial starter culture of the invention.

The invention also relates to a method for the production of bread, which comprises the step of adding a bacterial starter culture of the invention and/or the sourdough of the invention.

The invention also relates to bread containing the bacterial starter culture of the invention and/or the sourdough of the invention or produced using them or a mixture thereof.

The invention also relates to a method for the microbiological production of dextran, which comprises a step in which the bacterial strain Weissella cibaria of the invention and/or the composition of the invention are grown on a liquid medium without dextrose (without glucose) with the addition of saccharose, an organic source of nitrogen containing amino acids and short peptides, vitamins from the group B and mineral salts.

In a preferred method, the culture is carried out on MRS medium (DeMan-Rogosa-Sharpe broth) or another medium for lactic acid bacteria, preferably with the addition of 5-10%±2 by wt. of saccharose.

The invention also relates to a bacterial preparation comprising the bacterial strain Weissella cibaria of the invention and/or the composition of the invention for use as an active ingredient in a probiotic, a therapeutic preparation, a functional food, a dietary supplement, for use as an active ingredient in a drug intended for the prevention and/or treatment of intestinal cancer, preferably colon cancer.

The invention also relates to the use of the bacterial strain Weissella cibaria of the invention and/or the composition of the invention as a functional additive in a functional food, a functional additive in a food drink, as an active ingredient of a probiotic preparation, a bacterial preparation, a dietary supplement, a pharmaceutical composition.

The invention also relates to the use of the bacterial strain Weissella cibaria of the invention and/or the composition of the invention in the food industry as an emulsifier, thickener, stabilizer or texture improver of a food product.

The invention also relates to the use of the bacterial strain Weissella cibaria of the invention and/or the composition of the invention for the microbiological production of exopolysaccharides (EPS), preferably dextran.

The invention also relates to the use of the bacterial strain Weissella cibaria of the invention and/or the composition of the invention and/or the bacterial starter culture of the invention for the production of sourdough for bread.

The invention also relates to the use of the bacterial strain of Weissella cibaria of the invention and/or the composition of the invention and/or the sourdough for making bread (bakery products) of the invention for the production of bread (bakery products).

The strain of Weissella cibaria IBB3394 deposited as KKP 2094p, which is the object of the invention, has preferable technological and functional properties and is an advantage over currently known and used strains. This strain, similarly to the previously described Weissella confusa/cibaria EPS_3-IBB3326 strain (Buksa et al. 2021), with high EPS production efficiency, was isolated from bakery sourdough and, based on analogous results obtained by the inventors, has the ability to produce EPS (dextran) at the level of 50 g/l (unpublished data). The amount of EPS produced by the IBB3326 and IBB3394 strains is higher than that described for other lactic acid bacteria strains, except for the W. confusa VP30 strain producing dextran at the level of 60 g/l (Jin et al., 2019). However, it should be emphasized that the yield of raw EPS for the VP30 strain was not comparable due to the significant content of impurities. The above-described strains of W. cibaria 27 and W. confusa VP30 with high dextran production efficiency were isolated from kimchi and child feces, respectively, and may not be adapted to the sourdough environment, where a preferable effect of EPS on the quality of sourdough bread has previously been demonstrated. In addition to the ability to produce dextran, the strain W. cibaria IBB3394 (KKP 2094p) is also characterized by other demonstrated functional properties (probiotic properties): antimicrobial and anticancer activity, exceptionally high resistance to bile salts and low pH compared to other strains of Weissella confusa/cibaria isolated from various sources tested by the inventors and the presence of all genes of the vitamin B2 and K2 production pathway as well as genes of the vitamin B1 recovery pathway. None of the probiotic Weissella cibaria strains described in the literature shows such high dextran production efficiency, antimicrobial and anticancer activity and useful physicochemical properties as the selected strain of W. cibaria IBB3394 (KKP 2094p) of the invention. Moreover, it should be emphasized that in the case of W. cibaria IBB3394 (KKP 2094p) of the invention, the probiogenomic analysis showed antibiotic resistance at a level consistent with the values recommended by EFSA, while confirming the lack of antibiotic resistance genes and the lack of virulence genes. The lack of cytotoxicity of the exopolysaccharide produced by the strain W. cibaria IBB3394 (KKP 2094p) on the tested cell lines was also demonstrated: human intestinal epithelium (colorectal adenocarcinoma) Caco-2 (ATCC HTB-37) and human embryonic kidney epithelial cells HEK293 (ATTC CRL-1573) and anticancer properties of W. cibaria IBB3394 (KKP 2094p) have been demonstrated, especially against intestinal cancer, especially the colon. The essence of the invention is the strain of lactic acid bacteria Weissella cibaria IBB3394 (KKP 2094p) with a high exopolysaccharide production efficiency, antimicrobial and anticancer activity, excellent acidifying properties, very good survival in conditions imitating the digestive tract, characterized by the presence of all genes of selected vitamin production or recovery pathways, with safety proven.

The strain Weissella cibaria (for which the own designation IBB3394 is used in the present description) was deposited on Feb. 6, 2023 under the deposit no. KKP 2094p as a deposit for patent purposes in IAFB collection (KKP collection) (Culture Collection of Industrial Microorganisms, prof. Waclaw Dabrowski IAFB-SRI) i.e. in the IAFB-SRI Collection of Industrial Microorganisms of prof. Waclaw Dabrowski (IBPRS-PIB Instytut Biotechnologii Przemysłu Rolno-Spożywczego im. prof. Wacława Dąbrowskiego-Państwowy Instytut Badawczy w Warszawie, Polska, IAFB-Institute of Agricultural and Food Biotechnology of prof. Waclaw Dabrowski—State Research Institute in Warsaw, Poland, IAFB-SRI).

A new, unknown and previously undescribed strain of lactic acid bacteria W. cibaria KKP 2094p of the invention shows preferable technological properties for the production of sourdoughs and sourdough bread as well as probiotic and anticancer properties, is characterized by exceptionally high dextran production efficiency, has antimicrobial activity against S. aureus and S. epidermidis and against spore-forming bacteria undesirable in bread (antagonistic activity against Bacillus cereus, Bacillus brevis and unique against Lysynibacillus fusiformis), inhibits the proliferation of colon cancer cells, is highly resistant to bile salts and low pH, has all the genes of the vitamin B2 and K2 production pathway as well as the genes of the vitamin B1 recovery pathway, and, moreover, shows an acceptable level of antibiotic resistance. The probiogenomic analysis of this strain did not detect antibiotic resistance or virulence genes, which confirms its safety.

The strain W. cibaria KKP 2094p was isolated from bakery sourdough.

The invention also relates to the use of the strain for the biotechnological production of dextran.

Preferably, the culture medium for W. cibaria KKP 2094p should include saccharose, an organic source of nitrogen containing amino acids and short peptides, B vitamins and necessary mineral salts.

For example, a suitable medium for culturing bacteria of the genus Weissella sp. enabling the production of EPS is the commercial MRS medium (DeMan-Rogosa-Sharpe broth) with the addition of saccharose.

In a preferred embodiment of the dextran production method, the culture of W. cibaria KKP 2094p is performed on MRS medium without dextrose (without glucose) with the addition of saccharose, preferably saccharose at a concentration of 10 wt. %, allowing for the maximum efficiency of dextran production.

The invention relates to a method of obtaining dextran from the culture of the strain W. cibaria KKP 2094p of the invention.

Preferably, the obtained cultures were diluted with cold, sterile water (1:1, v/v), the post-culture fluid was separated from the cells by centrifugation (15 151 rcf, 4° C., 15 min), and then EPS was extracted in the obtained supernatant. EPS precipitation was carried out in two stages, each time adding 4 volumes of cooled 75% ethanol. After the first step, the sample was centrifuged, the sediment was suspended in water and a second portion of ethanol was added.

The invention also relates to the use of the strain W. cibaria KKP 2094p, due to the preferable technological properties demonstrated by this strain, for the production of sourdoughs and sourdough bread (dextran production, antagonistic activity against spore-forming bacteria undesirable in bread).

Antagonistic activity was tested against spore-forming bacteria undesirable in bread (Bacillus cereus, Bacillus brevis and Lysynibacillus fusiformis).

The invention also relates to the use of the strain due to its probiotic properties.

In preferred embodiments, the strain W. cibaria KKP 2094p showed antagonistic activity against S. aureus and S. epidermidis strains, but did not inhibit the growth of a strain representing the lactic acid bacteria L. lactis.

Antagonistic activity of overnight culture of the strain W. cibaria KKP 2094p, its cell suspension in PBS and the post-culture supernatant of this strain was demonstrated against S. aureus IBB4005 (i.e. multi-resistant S. aureus strain NCTC9789 [PS80] showing resistance to AMP Pen Asa Cad Mer). The most preferred activity was observed for overnight culture and cell suspension in PBS.

It has been shown that the strain W. cibaria KKP 2094p has features considered desirable for probiotic bacteria: resistance to low pH, ensuring the passage of bacteria to further sections of the digestive tract; resistance to bile salts, ensuring the survival of bacteria in the presence of pancreatic juices.

It has been shown that the dextran produced by the strain W. cibaria KKP 2094p is not cytotoxic to the human Caco-2 cell lines and non-cancerous renal epithelial cells HEK293, nor does it show antagonistic activity against the tested strains of S. aureus and L. lactis bacteria.

It was shown that the strain W. cibaria KKP 2094p inhibited the proliferation of colon cancer cells, and the demonstrated anticancer activity is stronger than the activity of known strains of lactic acid bacteria (Lactococcus lactis deposited in the PCM as B/00311 and B/00312) with confirmed antiproliferative activity (patent no. PL241568B1, Sałański et al., 2022).

The strain W. cibaria KKP 2094p was sequenced and the probiogenomic analysis confirmed the absence of antibiotic resistance and virulence genes, thus confirming the possibility of its probiotic and medical use.

Bioinformatic analysis of the strain W. cibaria KKP 2094p was performed in accordance with the EFSA recommendations using recommended programs and databases.

In this application, the term “biomass” is to be understood as the biomass of cells of the bacterial Weissella cibaria KKP 2094p strain obtained as a result of cell multiplication in liquid media or on solid media with a composition adapted to the requirements of lactic acid fermentation bacterial cultures.

In this application, the term “potentially probiotic” is to be understood as features demonstrated in in vitro tests with a possible beneficial effect on the host's health in accordance with available literature data, in particular: EPS production, activity against S. aureus and S. epidermidis bacteria, lowering the environmental pH, resistance to low pH and the presence of bile salts.

In this application, the term “antimicrobial activity” is to be understood as an antagonistic effect against S. aureus and S. epidermidis bacteria and against spore-forming bacteria undesirable in bread (Bacillus cereus, Bacillus brevis and Lysynibacillus fusiformis).

In this application, the term “confirmed safety” is to be understood as the permissible level of resistance to the 9 antibiotics recommended by EFSA: ampicillin (AM), chloramphenicol (CL), clindamycin (CM), kanamycin (KM), gentamicin (GM/CN), streptomycin (SM), tetracycline (TC), vancomycin (VA), erythromycin (EM), and the lack of antibiotic resistance genes and the lack of virulence genes in probiogenomic analysis performed in accordance with EFSA recommendations using recommended programs and databases.

In this application, the term “cytotoxic” is understood to mean cytotoxicity above 10% and resulting cell survival below 90% according to the values proposed by EL-Adawi et al. (2012) for EPS produced by lactic acid bacteria.

In this application, the term “overnight culture” is to be understood as a culture of a bacterial strain established from a single colony in an appropriate medium, incubated for several hours.

In this application, the term “supernatant” is to be understood as the post-culture fluid separated from bacterial cells.

In this application, the term “cell extract” is to be understood as the fluid obtained after disruption using glass beads of bacterial cells separated by centrifugation from an overnight culture and suspended in PBS buffer.

In this application, the term “cell suspension in PBS” is to be understood as bacterial cells obtained from overnight culture by centrifugation, washed and resuspended in PBS buffer.

### DETAILED DESCRIPTION OF THE INVENTION

The strain Weissella cibaria IBB3394 deposited as KKP 2094p of the invention was isolated as shown in Example 1 by searching for strains of Weissella sp. capable of producing exopolysaccharides (EPS) originating from various environments (plants, fermented plants, fermented dairy products). The strain W. cibaria KKP 2094p was identified as Weissella confusa/cibaria by sequencing the 16S rRNA genes. The species affiliation of the strain W. cibaria KKP 2094p was then determined as Weissella cibaria by genome sequencing and mass spectroscopy using a MALDI TOF apparatus. The strain W. cibaria KKP 2094p was selected from five isolated strains of Weissella sp., also compared to strains of this type previously isolated from spontaneous bakery sourdoughs, based on the results of the analysis classifying it into the group of strains with the highest antagonistic activity against S. aureus (FIG. 1) and at the same time confirming the lack of such activity against the representative of lactic acid bacteria (FIG. 2), carried out as described in Example 2 and exceptionally high resistance to low pH and the presence of bile salts compared to the other tested strains (FIG. 3), under the conditions used in Example 3 to check the survival of strains in conditions imitating the gastrointestinal tract. For the strain W. cibaria KKP 2094p, antagonistic activity against other S. aureus strains and S. epidermidis strains was confirmed (Example 4). This example also demonstrated the ability of the strain W. cibaria KKP 2094p to acidify the environment. The possible use of the strain W. cibaria KKP 2094p as a potentially probiotic strain with activity against S. aureus has been demonstrated (FIG. 4) and confirmed in Example 5 for both the overnight culture of the strain and cell suspension in PBS. This type of activity, but at a lower level, was observed for the supernatant, which indicates that the inhibitory effect on S. aureus is not solely caused by lowering the pH of the environment. Moreover, the lack of antagonistic activity in the case of the cell extract indicates an important, as yet unknown role played by living cells of this strain. In Example 6, the strain W. cibaria KKP 2094p was shown to have an exceptionally strong inhibitory effect on the proliferation of colorectal cancer cells (FIG. 5), indicating the possibility of using the strain in the treatment of gastrointestinal cancer.

The souring ability observed in Example 4 for the strain W. cibaria KKP 2094p of the invention, isolated from bakery sourdough (adapted to live in such an environment), as well as the antagonistic activity against spore-forming bacteria undesirable in sourdoughs and bread, demonstrated in Example 7 (Bacillus cereus, Bacillus brevis and Lysynibacillus fusiformis) confirm the possibility of using the strain W. cibaria KKP 2094p due to its preferable technological properties for the production of sourdoughs and sourdough bread. The use of this strain for this purpose is also preferable due to its ability to produce EPS, which plays an important role in shaping the rheological and structural properties of bread.

A selected strain of W. cibaria KKP 2094p with the ability to produce EPS was used in Example 8 to produce EPS, obtaining one of the highest documented biotechnological yields of dextran production on a medium with 10% saccharose.

In Example 9, a freshly obtained EPS and EPS stored under refrigerated conditions for 6 months were checked for antagonistic activity against S. aureus and a lactic acid bacteria strain (FIG. 6). The EPS concentration range used from 0 to 20 mg/ml did not significantly inhibit the growth of any of the indicator strains, thus indicating that such EPS does not have significant antagonistic activity towards the tested bacterial strains.

Exopolysaccharides used as food and feed additives should be safe for humans and animals. The cytotoxicity of exopolysaccharides produced by lactic acid bacteria should not be higher than 10%, which is to be understood as that the percentage of surviving cells should not be lower than 90%. (EL-Adawi et al., 2012). The cytotoxicity of the exopolysaccharide from the strain Weissella cibaria (KKP 2094p) was tested as described in Example 10 against two human cell lines derived from the American Type Culture Collection: Caco-2 colorectal adenocarcinoma (ATCC HTB-37) and non-neoplastic cells, HEK293 renal epithelial cells (ATTC CRL-1573). Treatment of cell lines with exopolysaccharide concentrations from 2.5 to 30% did not result in statistically significant inhibition of proliferation, thus confirming the lack of cytotoxicity of the tested EPS. (FIG. 7-10).

Analyzes confirming safety were also performed for the strain. In Example 11, antibiotic susceptibility testing confirmed the level of resistance at an acceptable level according to EFSA guidelines. In the case of the genus Weissella, for which no specific limit values are given, the results, according to the recommendation, should be compared with the results for the most phylogenetically related bacteria (heterofermentative lactobacilli and Leuconostoc). Additionally, the safety of the strain (no antibiotic resistance genes and no virulence factors) was confirmed in the probiogenomic analysis described in Example 12.

In Example 13, the strain W. cibaria KKP 2094p was successfully used to produce sourdough bread, which scored better compared to a bread obtained without this strain.

### BRIEF DESCRIPTION OF THE DRAWINGS

Examples of embodiments of the invention are presented in the figures of the drawing, on which:

FIG. 1 Shows the result of testing the antagonistic activity of Weissella sp. strains against S. aureus IBB4005. Counting from left to right, top to bottom, the plate (a) contains: 3714 (1), 3715 (2), 3716 (3), 3717 (4), 3277 (5), 3278 (6), 3279 (7), 3385 (8), 3386 (9), 3387 (10), 3388 (11), 3389 (12), 3393 (13), 3394 (14) (i.e. W. cibaria IBB3394 (KKP 2094p) and K-control strain (L. lactis IBB1339 producing nisin). While, the plate (b) contains: 3287 (15), 3325 (16), 3326 (17), 3327 (18), 3382 (19), 3383 (20), 3384 (21), 3280 (22), 3281 (23), 3282 (24) 3284 (25), 3285 (26), 3286 (27) and K-control strain (L. lactis IBB1339 producing nisin).

FIG. 2 Shows the result of testing the antagonistic activity of Weissella sp. strains against L. lactis IL1403. Counting from left to right, top to bottom, the plate (a) contains: 3714 (1), 3715 (2), 3716 (3), 3717 (4), 3277 (5), 3278 (6), 3279 (7), 3385 (8), 3386 (9), 3387 (10), 3388 (11), 3389 (12), 3393 (13), 3394 (14) (i.e. W. cibaria IBB3394 (KKP 2094p) and K-control strain (L. lactis IBB1339 producing nisin). While, the plate (b) contains: 3287 (15), 3325 (16), 3326 (17), 3327 (18), 3382 (19), 3383 (20), 3384 (21), 3280 (22), 3281 (23), 3282 (24) 3284 (25), 3285 (26), 3286 (27) and K-control strain (L. lactis IBB1339 producing nisin).

FIG. 3 Shows the response of Weissella sp. strains to stress associated with the presence of bile salts and low pH.

FIG. 4 Shows the effect of W. cibaria IBB3394 (KKP 2094p) (overnight culture, supernatant, cell extract, live cells suspended in PBS buffer) on the survival of S. aureus IBB4005. The number of Staphylococcus bacteria expressed as a percentage was presented relative to the number in the control in MRS (for culture and supernatant) or PBS (for cells and extract). Calculations for measurement after the start (T₀), after one hour (T₁ₕ) and three hours (T₃ₕ) of incubation.

FIG. 5 Shows the inhibitory effect of the Weissella sp. IBB3394 (KKP 2094p) strain on the proliferation of Caco-2 colorectal adenocarcinoma cells compared to the effect of two other strains of lactic acid bacteria, including the IBB109 strain with previously confirmed antiproliferative activity. Proliferation of Caco-2 cells incubated with bacterial strains was measured relative to bacteria-free Caco-2 cell culture (100%) using the BrdU colorimetric assay. Error bars represent the standard error.

FIG. 6 Shows the result of testing the effect of EPS produced by Weissella sp. IBB3394 (KKP 2094p) on the growth of S. aureus IBB4005 (a) and L. lactis IL1403 (b). Fresh EPS and EPS samples stored for 6 months under refrigerated conditions (20, 10, 5, 2.5, 0 mg/ml) were dropped above the line, while positive controls—with bacteriocin nisin in various concentrations (100, 50, 20, 10, 5, 2.5 mg/ml), lower part of the plate from left to right.

FIG. 7 Shows the result of the study of cell survival of the Caco-2 colorectal adenocarcinoma cell line after incubation with various concentrations of exopolysaccharide isolated from the culture of the Weissella sp. IBB3394 strain (KKP 2094p) in relation to the cell line not co-incubated with exopolysaccharide. Error bars represent the standard error.

FIG. 8 Shows the result of testing the survival of the HEK293 kidney epithelial cell line after incubation with various concentrations of exopolysaccharide isolated from the culture of the Weissella sp. IBB3394 strain (KKP 2094p) in relation to the cell line not co-incubated with exopolysaccharide. Error bars represent the standard error.

FIG. 9 Shows the result of cytotoxicity testing of exopolysaccharide isolated from the culture of the Weissella sp. IBB3394 strain (KKP 2094p) on cells of the Caco-2 colorectal adenocarcinoma cell line in relation to the cell line not co-incubated with exopolysaccharide. Error bars represent the standard error.

FIG. 10 Shows the result of cytotoxicity testing of exopolysaccharide isolated from the culture of the Weissella sp. IBB3394 strain (KKP 2094p) on the HEK293 kidney epithelial cell line in relation to the cell line not co-incubated with exopolysaccharide. Error bars represent the standard error.

The publications cited herein and the references provided therein are hereby incorporated by reference in their entirety. The following examples illustrate the invention without limiting it in any way.

### DESCRIPTION OF EMBODIMENTS

The following examples were presented merely to illustrate the invention and to clarify its various aspects, but are not intended to be limitative, and should not be equated with all its scope, which is defined in the appended claims.

### EXAMPLES

### Example 1 Isolation and Identification of EPS-Producing Strains

The search for bacterial strains capable of producing EPS was carried out on a solid MRS selection medium without dextrose with the addition of 10% saccharose, on which sourdough, fermented cabbage and cucumber juice, and liquid obtained after thoroughly rinsing plant fragments in PBS buffer (pH 7.4), or after suspending fermented milk products were seeded by streak plate method. The plates were incubated in aerobic conditions at 30° C. for a minimum of 48 hours. Single colonies of bacteria with mucoid morphology were passaged onto solid MRS medium to obtain pure cultures of isolated bacteria, which strains were deposited in the IBB PAN Collection (COLIBB, Poland).

Species identification was determined by sequencing the 16S rRNA genes. Among the EPS-producing bacteria isolated from the saccharose-containing medium, bacteria belonging to Leuconostoc sp. (12 strains) and Weissella confusa/cibaria (5 strains) were detected. At a further stage of research, after determining the surprising functional properties of the W. cibaria IBB3394 strain, its species affiliation was confirmed as Weissella cibaria by genome sequencing and mass spectroscopy using a MALDI TOF apparatus when depositing this strain to the IAFB (KKP) (IAFB Collection of Industrial Microorganisms of prof. Waclaw Dabrowski) where it was deposited as KKP 2094p.

### Example 2 Screening of the Weissella sp. Strains for Antagonistic Activity Against the Pathogenic S. aureus Strain

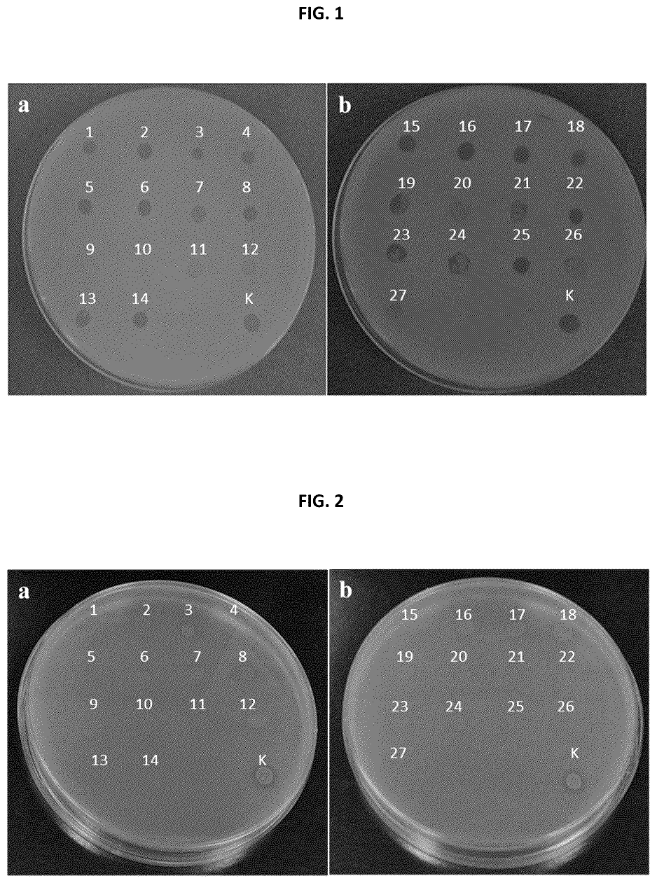

Antagonistic activity of 5 strains of Weissella sp. isolated as described in Example 1 and 22 strains previously isolated from spontaneous bakery sourdoughs (Table 1) against the pathogenic S. aureus strain IBB4005 (FIG. 1) and the L. lactis IL1403 strain representing the lactic acid bacteria (FIG. 2) was analyzed by dripping onto a plate with the indicator strain, against the nisin-producing L. lactis IBB1339 strain as a positive control.

TABLE 1
Weissella confusa/cibaria strains used in the example.

| Number | Insulation source | Number in “COLIBB” |
|---|---|---|
| 1 | low-salt cucumbers pickles | 3714 |
| | (“Zieleniak” bazaar) | |
| 2 | low-salt cucumbers pickles | 3715 |
| 3 | arugula | 3716 |
| 4 | soybeans (dry) | 3717 |
| 5 | rye sourdough | 3277 |
| 6 | rye sourdough | 3278 |
| 7 | rye sourdough | 3279 |
| 8 | common wheat sourdough | 3385 |
| 9 | common wheat sourdough | 3386 |
| 10 | common wheat sourdough | 3387 |
| 11 | common wheat sourdough | 3388 |
| 12 | common wheat sourdough | 3389 |
| 13 | common wheat sourdough | 3393 |
| 14 | bakery sourdough | 3394 |
| 15 | rye sourdough | 3287 |
| 16 | spelled wheat sourdough | 3325 |
| 17 | spelled wheat sourdough | 3326 |
| 18 | spelled wheat sourdough | 3327 |
| 19 | common wheat sourdough | 3382 |
| 20 | common wheat sourdough | 3383 |
| 21 | common wheat sourdough | 3384 |
| 22 | rye sourdough | 3280 |
| 23 | rye sourdough | 3281 |
| 24 | rye sourdough | 3282 |
| 25 | rye sourdough | 3284 |
| 26 | rye sourdough | 3285 |
| 27 | rye sourdough | 3286 |

Weissella sp., L. lactis IBB1339 (control strain) and IL1403 (indicator strain) strains from the IBB PAN strain collection (COLIBB, Poland) were streaked on plates with MRS, GM17 and BHI medium, respectively, and incubated for 48 hours at 30° C. S. aureus IBB4005 (indicator strain) was also plated on BHI medium (BSL2 laboratory). A single colony from each plate was inoculated into liquid medium and incubated overnight. Working cultures of bacteria were grown in MRS broth at 30° C. for Weissella, in GM17 liquid medium at 30° C. for L. lactis IBB1339 and in BHI broth under aerobic conditions at 37° C. with shaking and at 30° C. without shaking for indicator strains, S. aureus IBB4005 and L. lactis IL1403, respectively. Soft BHI agar heated to 55° C. (5 ml) was inoculated with 100 μl of overnight culture (indicator strains: S. aureus IBB4005, L. lactis IL1403) and immediately poured onto a large BHI solid medium plate and spread evenly to cover the surface. Following agar setting, 5 μl of overnight cultures of the tested Weissella sp. cultures and the positive control (nisin-producing strain L. lactis IBB1339) were applied to specific places on the plate. The plates were allowed to absorb the drops and incubated overnight at 37° C. and 30° C. for S. aureus IBB4005 and L. lactis IL1403, respectively. The experiment was performed in three biological replicates. Antagonism towards indicator strains was assessed by observing growth inhibition zones.

The size and transparency of such zones vary depending on the strain. Overall, four different effects were observed: i.) clear inhibition zones, ii.) inhibition zones with visible growth of the test strain in the center, iii.) blurred/indistinct zones with a faint increase in the index and visible growth of the test strain in the center, and iv.) no inhibition zones growth around the tested strain. The first two effects were classified as strong antagonistic activity and marked as (+), the third effect was bacteriostatic (+/−), and the last effect was characterized as no antagonistic activity (−). The result obtained from three replicates (Table 2) indicated that some Weissella sp. strains were able to inhibit the growth of S. aureus, some were bacteriostatic, while others did not inhibit the growth of S. aureus. At the same time, none of the tested strains inhibited the growth of the lactic acid bacteria L. lactis IL1403 strain (Table 2).

TABLE 2
Antagonist activity test results.

| | | Antagonistic activity against | | | | | |
|---|---|---|---|---|---|---|---|
| | | L. lactis IL1403 | | | S. aureus IBB4005 | | |
| | Number in | Rep. | Rep. | Rep. | Rep. | Rep. | Rep. |
| Number | “COLIBB” | 1 | 2 | 3 | 1 | 2 | 3 |
| 0 | 1339 | + | + | + | + | + | + |
| 1 | 3714 | − | − | − | + | + | + |
| 2 | 3715 | − | − | − | + | + | + |
| 3 | 3716 | − | − | − | + | + | + |
| 4 | 3717 | − | − | − | + | + | + |
| 5 | 3277 | − | − | − | + | + | + |
| 6 | 3278 | − | − | − | + | +/− | +/− |
| 7 | 3279 | − | − | − | + | + | + |
| 8 | 3385 | − | − | − | + | + | + |
| 9 | 3386 | − | − | − | − | − | − |
| 10 | 3387 | − | − | − | − | − | − |
| 11 | 3388 | − | − | − | +/− | + | + |
| 12 | 3389 | − | − | − | +/− | +/− | +/− |
| 13 | 3393 | − | − | − | + | + | + |
| 14 | 3394 | − | − | − | + | + | + |
| 15 | 3287 | − | − | − | + | +/− | +/− |
| 16 | 3325 | − | − | − | + | + | + |
| 17 | 3326 | − | − | − | + | + | + |
| 18 | 3327 | − | − | − | + | +/− | + |
| 19 | 3382 | − | − | − | + | +/− | +/− |
| 20 | 3383 | − | − | − | +/− | +/− | +/− |
| 21 | 3384 | − | − | − | + | + | + |
| 22 | 3280 | − | − | − | + | +/− | +/− |
| 23 | 3281 | − | − | − | + | + | +/− |
| 24 | 3282 | − | − | − | + | + | + |
| 25 | 3284 | − | − | − | + | +/− | +/− |
| 26 | 3285 | − | − | − | +/− | + | +/− |
| 27 | 3286 | − | − | − | +/− | + | +/− |

### Example 3. Survivability Test of Weissella sp. Strains at Low pH and in the Presence of Bile Salts

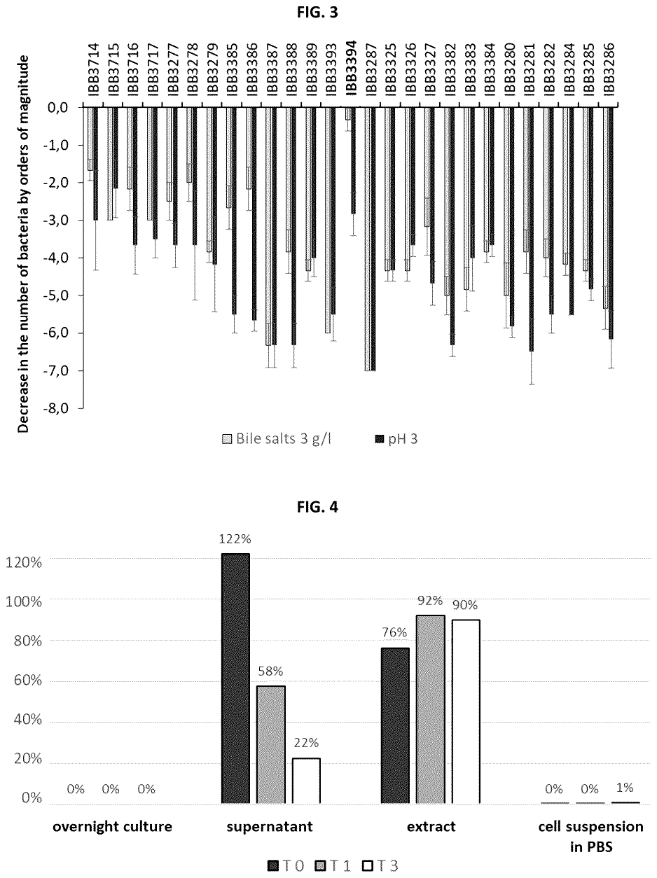

One of the important defining properties of probiotic bacteria is their ability to survive in the digestive tract. The resistance of 5 Weissella sp. strains isolated as described in Example 1 and 12 strains previously isolated from spontaneous sourdoughs, to bile salts (bile salt stress) and low pH (acid stress) was tested as follows. Freshly grown colonies on IST-MRS medium [90% ISO-Sensitest broth (Oxoid) and 10% MRS (Merck)] for each bacterial isolate were suspended in PBS, pH=7.4 (PBS404, BioShop) and diluted to OD600ₙₘ=0.5 in 1 ml of PBS, pH=7.4 (control); PBS, pH=3 (acid stress); and PBS, pH=7.4, containing 3 g/l of two bile salts: sodium cholate and sodium deoxycholate (B8756, Sigma-Aldrich) (bile salt stress). Samples were incubated for one hour at 37° C. After incubation, the tubes were centrifuged (3 minutes, 4° C., 10,000 rpm) and the supernatant was poured off, and the cell pellet was suspended in 0.5 ml of PBS buffer pH 7.4. Dilutions ranging from 10⁻¹ to 10⁻⁶ were prepared from each sample. 20 μl of dilutions were spotted on IST-MRS solid substrate plates. The plates were incubated for 48 h at 30° C. under aerobic conditions. Screening experiments for strain characterization under conditions mimicking the gastrointestinal tract were performed in three biological replicates, and the mean for each condition was calculated. Susceptibility to stress factors was expressed as an order of magnitude decrease in the number of bacterial colony-forming units (CFU)/ml compared to control conditions (pH PBS=7.4). The results obtained are presented in a chart (FIG. 3), based on them, the isolate IBB3394 (subsequently deposited as KKP 2094p) was selected as an unique strain, the most resistant to bile salts and low pH.

### Example 4. Testing of the Selected Weissella sp. Strains for Antagonistic Activity Against S. aureus and S. epidermidis Strains

Six strains of lactic acid bacteria of the genus Weissella (confusa/cibaria) selected from the Collection of Bacterial Strains of the IBB PAN (COLIBB) were tested for:
acidifying activity, in order to examine the possible impact of lowering pH on inhibiting the growth of S. aureus and S. epidermidis strains,
antagonistic activity against living cells of three pathogenic strains of S. aureus, including a multi-resistant strain,
antagonistic activity towards living cells of two strains of S. epidermidis representing a species of bacteria causing opportunistic infections (not causing infections for healthy people).

The tested Weissella strains spotted on a solid BHI medium with bromocresol purple (acidity indicator) caused a change in the color of the medium from violet-purple to yellow, which indicates the acidification of the medium around the drop zone. The pH values of the overnight cultures ranged from 4.23 to 4.89.

The antagonistic activity test was performed by dropping 5 μl of liquid overnight cultures of Weissella strains onto large plates (BHI with bromocresol purple) with the indicator strains S. aureus IBB4002, IBB4005 and IBB4009 and S. epidermidis 6PII6 and 5L03 inoculated in the form of lawn. The plates were incubated for 24 hours at 37° C. Growth inhibition zones were observed around the tested Weissella strains for all indicator strains of S. aureus (Table 3) and S. epidermidis (Table 4).

TABLE 3
Results of testing for antagonistic activity against S. aureus strains (average of two biological replicates).

| | | drop | | | drop | | | drop | | |
|---|---|---|---|---|---|---|---|---|---|---|
| | | diameter | diameter | | diameter | diameter | | diameter | diameter | |
| | | on BHI | of the | | on BHI | of the | | on BHI | of the | |
| | | with | inhibition | | with | inhibition | | with | inhibition | |
| | | purple | zone of | | purple | zone of | | purple | zone of | |
| | | after | S. aureus | | after | S. aureus | | after | S. aureus | |
| | | 24 h in | 4002 on | difference | 24 h in | 4005 on | difference | 24 h in | 4009 on | difference |
| | number in | a 4002 | BHI with | for 4002 | a 4005 | BHI with | for 4005 | a 4009 | BHI with | for 4009 |
| no | “COLIBB” | plate | purple | [mm] | plate | purple | [mm] | plate | purple | [mm] |
| 1 | 3287 | 7.50 | 9.50 | 2.00 | 8.00 | 9.50 | 1.50 | 7.75 | 9.50 | 1.75 |
| 2 | 3325 | 7.75 | 10.00 | 2.25 | 6.75 | 9.25 | 2.50 | 7.00 | 9.00 | 2.00 |
| 3 | 3326 | 7.25 | 10.25 | 3.00 | 7.75 | 10.25 | 2.50 | 7.50 | 9.00 | 1.50 |
| 4 | 3389 | 7.50 | 9.70 | 2.20 | 7.25 | 8.95 | 1.70 | 7.25 | 9.75 | 2.50 |
| 5 | 3393 | 7.25 | 9.05 | 1.80 | 7.50 | 9.55 | 2.05 | 7.25 | 9.75 | 2.50 |
| 6 | 3394 | 7.50 | 9.50 | 2.00 | 7.75 | 9.50 | 1.75 | 7.25 | 8.75 | 1.75 |

TABLE 4
Results of testing for antagonistic activity against S. epidermidis
strains (average of two biological replicates).

| | | drop diameter | | | | | |
|---|---|---|---|---|---|---|---|
| | | on BHI with | diameter of the | | drop diameter | diameter of the | |
| | | purple after | inhibition zone of | | on BHI with | inhibition zone of | |
| | | 24 h in a | S. epidermidis | | purple after | S. epidermidis | |
| | number in | plate with | 6PII6 on BHI | difference | 24 h in a | 5L03 on BHI | difference |
| no | “COLIBB” | 6PII6 | with purple | for 6PII6 | 5L03 plate | with purple | for 5L03 |
| 1 | 3287 | 9.0 | 11.0 | 2.0 | 7.0 | 8.0 | 1.0 |
| 2 | 3325 | 8.8 | 10.0 | 1.2 | 7.0 | 9.0 | 2.0 |
| 3 | 3326 | 8.8 | 10.3 | 1.5 | 6.8 | 8.5 | 1.7 |
| 4 | 3389 | 9.3 | 12.0 | 2.7 | 7.0 | 8.0 | 1.0 |
| 5 | 3393 | 8.8 | 10.0 | 1.2 | 7.0 | 7.5 | 0.5 |
| 6 | 3394 | 8.8 | 10.0 | 1.2 | 7.0 | 8.5 | 1.5 |

### Example 5. Antagonistic Activity of the Strain W. cibaria IBB3394 (KKP 2094p) for Various Forms of Preparation (Cultures, Supernatants, Cell Extracts, Live Cells Suspended in PBS Buffer) Towards Live Cells of the S. aureus Strain

The experiment was performed for various variants:
Overnight culture of the strain W. cibaria IBB3394 (KKP 2094p)—900 μl,
Supernatant—900 μl of liquid overnight culture of W. cibaria IBB3394 (KKP 2094p)—centrifuged for 3 min, 13 thous. RPM, and then the supernatant was filtered through a 0.45 μm filter,
Extract from W. cibaria IBB3394 cells (KKP 2094p) sedimented by centrifugation (3 min, 13,000 RPM)—the pellet was suspended in 900 μl of PBS buffer and cells were disrupted with glass beads (3×1 min, with cooling breaks on ice after each 1 min), then centrifuged for 10 min 14 thous. RPM at 4° C. The liquid above the beads was collected and added to PBS (approx. 100 μl) to a final volume of 900 μl,
Cell suspension in PBS-900 μl of liquid overnight culture of W. cibaria IBB3394 (KKP 2094p) was centrifuged for 3 min, 13 thous. RPM, then the cell pellet was suspended in 900 μl of sterile PBS, centrifuged again for 3 min, 13 thous. RPM and the resulting precipitate was suspended in 900 μl of sterile PBS.

Dilution of overnight liquid cultures of the S. aureus indicator strain in sterile PBS IBB4005 (100000×).

Incubation of diluted Staphylococcus overnight cultures (100 μl from a 10⁻⁵ dilution) with individual variants for the W. cibaria IBB3394 strain and 900 μl of PBS or MRS medium as a control for 3 h at a temperature of approximately 30° C. Seeding 100 μl of each variant on LB plates in two repetitions immediately after the start of incubation (T₀), after one hour (T₁ₕ) and three hours (T₃ₕ). The plates were incubated at 42° C. (to eliminate the growth of LAB) for 24 h or 48 h (option with overnight culture and cells). The experiment was performed in 2 technical repetitions and a minimum of 2 biological repetitions. The results are presented in a graph (FIG. 4), in which the number of Staphylococcus bacteria expressed in percentages is presented in relation to the number in the control (in MRS or PBS).

A clear antagonistic effect was observed for overnight culture and cells suspended in PBS buffer. Moreover, the supernatant showed antistaphylococcal activity, while no such effect was observed for the cell extract.

### Example 6. Antiproliferative Activity of the Strain IBB3394 (KKP 2094p) Against Caco-2 Colorectal Adenocarcinoma Cells

The test of inhibition of colon cancer cell proliferation was performed according to the method described by Sałański et al. (2022), cultivating the Weissella sp. IBB3394 strain on MRS medium. BrdU assay results showed that strain IBB3394 strongly inhibited the proliferative activity of Caco-2 tumor cells (FIG. 5).

### Example 7. Testing of the Strain IBB3394 (KKP 2094p) for Antagonistic Activity Against Spore-Forming Bacteria Undesirable in Bread (Bacillus cereus, Bacillus brevis and Lysynibacillus fusiformis)

The antagonistic activity of strain IBB3394 (KKP 2094p) was tested against spore-forming bacteria spoiling bread according to the method described for strains isolated from rye sourdough (Litwinek et al., 2022). For this purpose, Luria-Bertani (LB) liquid medium (Difco Laboratories, Franklin Lakes, New Jersey, USA) was inoculated with spore-forming indicator bacteria (Bacillus cereus, Bacillus brevis and Lysynibacillus fusiformis) and cultured overnight. The overnight cultures were then used to inoculate the liquefied and cooled solid LB medium, which was immediately poured onto the plate. After the medium solidified, 5 μl of the tested strain grown on liquid MRS medium was placed on selected places on the plate. The plates were allowed to absorb the drops and incubated overnight at 30° C. Antagonism towards the strain was visualized by observing the growth inhibition zone. The presence of an inhibition zone was denoted as “++”, “+” or “+/−”, depending on size, while the absence of antagonistic activity was denoted as “−”. The obtained result was compared with the results obtained for Weissella sp. strains isolated from rye flour sourdoughs. (Litwinek et al., 2022). Based on the analysis, it was found that the IBB3394 strain (KKP 2094p) exhibits antagonistic activity rated “+” against all indicator strains, including a unique one against the Lysynibacillus fusiformis strain. This activity was higher than for strains isolated from rye sourdough, for which slightly weaker inhibition of Bacillus brevis growth was observed by all tested strains, antagonistic activity against Bacillus cereus for 4 out of 7 strains and no such activity against Lysynibacillus fusiformis.

### Example 8. Characterization of the Exopolysaccharide (EPS) Produced by IBB3394 (KKP 2094p)

The determination of the yield of exopolysaccharide (EPS) production by IBB3394 was performed for the strain grown on MRS medium without dextrose containing 5% or 10% saccharose. The production, followed by extraction, separation and characterization of EPS were performed according to the method described by Buksa et al. (2021). The results of the production yield analysis and the characteristics of EPS are presented in Table 5. The production yield of raw EPS for the IBB3394 strain was higher on the medium with 10% saccharose and amounted to nearly 50 g/l. As a result of the separation of raw EPS on SEC columns, a fraction with a high molar mass was detected, which was identified as dextran.

TABLE 5
Yield of crude exopolysaccharide (EPS) production and the percentage
of sugar residues after acid hydrolysis of the crude EPS preparation.

| | EPS production | | | | | |
|---|---|---|---|---|---|---|
| | yield in saccharose | Share of sugar residues after acid hydrolysis (%) | | | | |
| Strain | medium (g DM/I) | Glc | Gal | Man | Fru | Sum |

EPS produced in the presence of 5% saccharose in the production medium

| | | | | | | |
|---|---|---|---|---|---|---|
| IBB3394 | 26.4 ± 0.8 | 95.6 ± 0.8 | 0.4 ± 0.1 | 0.6 ± 0.8 | 0.3 ± 0.1a | 96.9 ± 0.8 |

EPS produced in the presence of 10% saccharose in the production medium

| | | | | | | |
|---|---|---|---|---|---|---|
| IBB3394 | 48.9 ± 0.9 | 94.9 ± 0.8 | 0.5 ± 0.2 | 0.5 ± 0.8 | 0.3 ± 0.2a | 96.2 ± 0.8 |

### Example 9. Analysis of the Effect of EPS Produced by Weissella sp. IBB3394 (KKP 2094p) on S. aureus IBB4005 and L. lactis IL1403

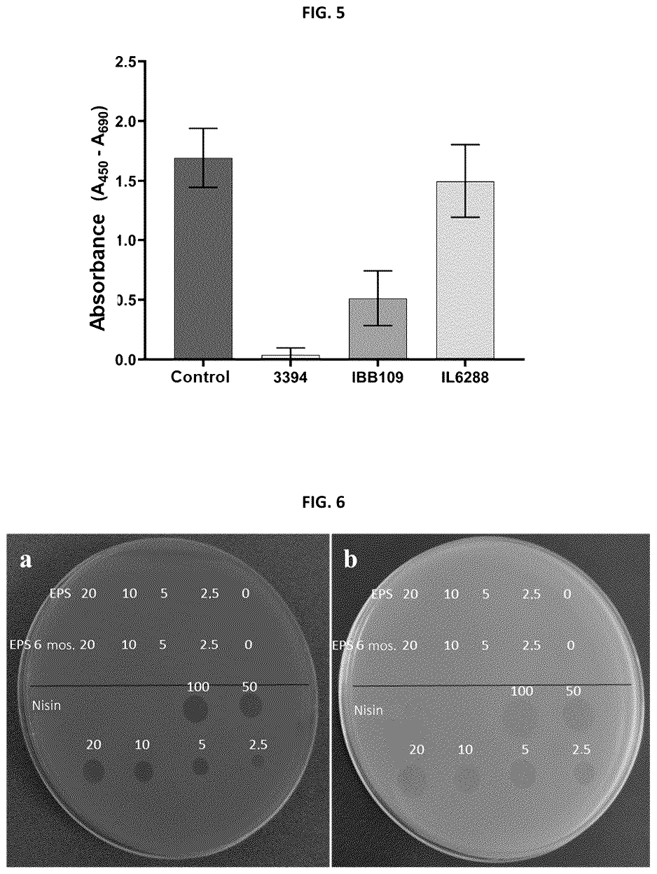

The assessment of the antagonistic activity of EPS produced by the IBB3394 strain was performed using a method similar to that described for the determination of the antagonistic activity of Weissella sp. strains in Example 2. In this case, specific concentrations (20, 10, 5, 2.5, 0 mg/ml) of fresh EPS and EPS stored for 6 months in refrigerated conditions were spotted on the plates with indicator strains against positive controls-nisin in various concentrations. (100, 50, 20, 10, 5, 2.5 mg/ml). The results obtained with different concentrations of nisin indicate that EPS from Weissella IBB3394 did not inhibit the growth of S. aureus at most of the tested concentrations (FIG. 6a), except for the concentration of 20 mg/ml, which showed very little inhibition. Furthermore, it was observed that there was no EPS concentration that would inhibit the growth of L. lactis IL1403 (FIG. 6b).

### Example 10. Cytotoxicity Test of Exopolysaccharide (EPS) Isolated from the Culture of Weissella sp. IBB3394 (KKP 2094p)

The study was conducted according to the method described by Vosough et al., 2021, with modifications. The Caco-2 adenocarcinoma cell line (ATCC HTB-37) and the HEK293 embryonic renal epithelial cell line cells (ATTC CRL-1573) were cultured under optimal conditions recommended by the ATCC, i.e. 37° C., 5% CO₂, 95% humidity. The medium (hereinafter referred to as cell line medium) used in the culture was MEM (Minimal Essential Medium; Gibco) enriched with 10% fetal bovine serum, amino acid solution (NEAA-non-essential amino acid solution) (1×), 1 mM sodium pyruvate, the addition of penicillin (100 U/ml) and streptomycin (100 μg/ml). Cell numbers of cell lines were determined in a Thoma chamber. The cells were diluted in the above-mentioned cell line medium to obtain a density of 10⁵ cells/ml and 100 μl of cell suspension per well were applied to a 96-well plate and incubated for 24 hours under optimal conditions. 100 μl of exopolysaccharide suspension with concentrations of 30, 20, 10, 5 and 2.5 mg/ml in the cell line medium was placed on 96-well plates containing Caco-2 or HEK293 cells after 24-hour culture, from which the medium had previously been collected. The plates were incubated for 24 hours at 37° C., 5% CO₂ and 95% humidity, and then the cell viability of the cell lines was assessed using a colorimetric method using the commercially available Cell Proliferation Kit I, MTT (Roche). Absorbance measurement was performed at wavelengths of 570 and 680 nm. The experiment was performed in 10 technical repetitions and 3 biological repetitions. The control consisted of cultures of Caco-2 and HEK293 cells treated with medium without exopolysaccharide. Survival was calculated using the following formula:

$$Survival [ \% ] = \frac{OD sample}{OD control} \cdot 100 \% ,$$

and cytotoxicity from the formula:

$$Cytotoxicity [ \% ] = 100 \% - Survival [ \% ] .$$

Due to the high viscosity of the exopolysaccharide suspension, the concentration of 30 mg/ml was chosen as the highest possible to use in this experiment. Statistical analyzes were performed using Shapiro-Wilk tests and multivariate analysis of variance (ANOVA).

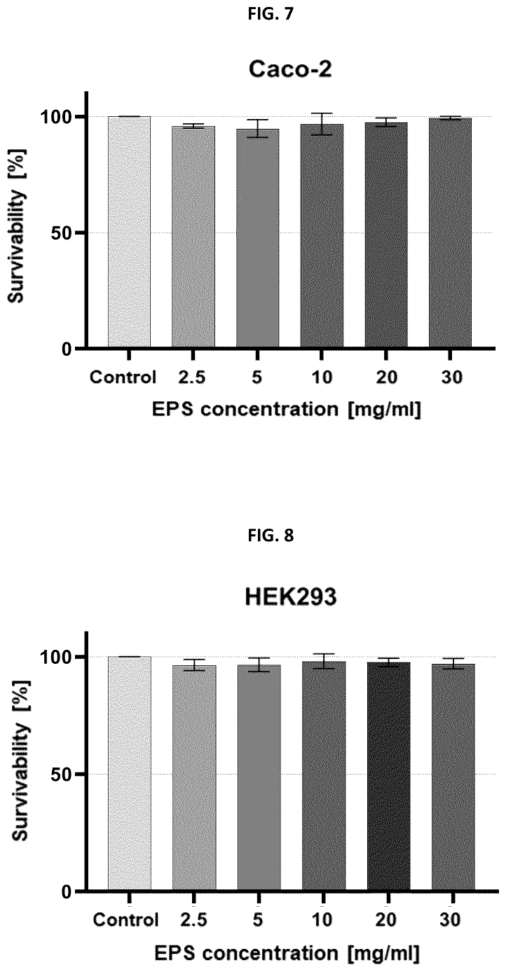

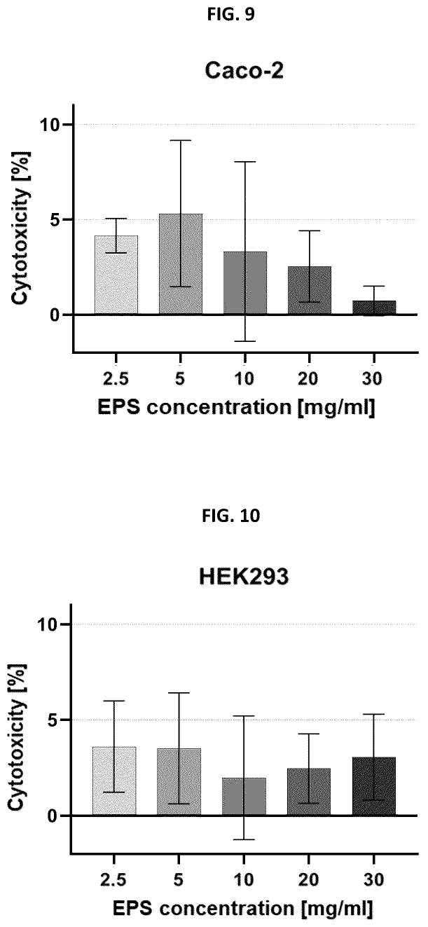

The results are shown in the figures (FIG. 7-10). The treatment of cell lines with exopolysaccharide concentrations from 2.5 to 30% did not result in statistically significant inhibition of proliferation, thus confirming the lack of cytotoxicity of the tested EPS.

### Example 11. Antibiotic Sensitivity Test

The safety assessment of the selected strain of Weissella sp. IBB3394 was carried out by testing antibiotic susceptibility using E-test strips (bioMerieux) in accordance with the manufacturer's recommendations. The tested strain was seeded on IST-MRS solid medium and incubated at 30° C. in aerobic conditions. The obtained colonies were used to prepare a bacterial suspension in PBS with OD600 nm=0.25—McFarland scale 1, which was then spread with a swab on the surface of the plates in different directions to ensure even distribution of the strain. Antibiotic strips were placed on the surface of the plate and the plates were incubated at 30° C. for 48 h in aerobic conditions. Resistance of the strain to 9 antibiotics was determined (in accordance with EFSA recommendations): ampicillin (AM), vancomycin (VA), gentamicin (GM), kanamycin (KM), streptomycin (SM), erythromycin (EM), clindamycin (CM), tetracycline (TC) and chloramphenicol (CL).

The results as obtained are presented in Table 6. It was shown that the minimum inhibitory concentration (MIC) for all tested antibiotics was below the limit values recommended by EFSA.

TABLE 6
Results of the sensitivity test of the IBB3394
strain (KKP 2094p) to antibiotics.

| | Minimum inhibitory | MIC breakpoints recommended | |
|---|---|---|---|
| | concentration | by EFSA [μg/ml] | |
| | (MIC) [μg/ml] | Heterofermentative | Leuconostoc |
| Antibiotic | IBB3394 | lactobacilli | spp. |
| AM | 0.38-0.5 | 2 | 2 |
| VA | >256 | * | * |
| GM | 4 | 16 | 16 |
| KM | 16 | 32 | 16 |
| SM | 8 | 64 | 64 |
| EM | 0.094 | 1 | 1 |
| CM | 0.016 | 1 | 1 |
| TC | 2 | 8 | 8 |
| CL | 2-3 | 4 | 4 |

* Not required (natural resistance)

### Example 12. Genome Analysis of the Strain IBB3394 (KKP 2094p)

DNA was isolated from the Weissella strain IBB3394, which was then subjected to whole genome sequencing at the DNA Sequencing and Synthesis Facility at IBB PAN using the Illumina MiSeq platform (Illumina, San Diego, USA) and the GridION sequencer (Oxford Nanopore Technologies, Oxford, Great Britain).

Various servers, software and databases were used for bioinformatic analysis. The RAST service was used to determine the coding sequences (CDS) and non-coding RNA (Aziz et al., 2008). The overall genomic sequence analysis of strain IBB3394 showed that the strain had a circular DNA chromosome of 2,488,843 bp and 44.8% GC content, while the number of total coding sequences (CDS) was 2,313. Analysis of the strain's genome sequence showed that it belongs to the species Weissella cibaria. The KEGG database (Kanehisa et al., 2022) and the automatic annotation service BlastKOALA were used for gene functional analysis (Kanehisa et al., 2016). The strain was found to encode 172 different metabolic pathways. The 30 complete pathway modules included, among others: complete vitamin biosynthetic pathways such as riboflavin (vitamin B2) and menaquinone (vitamin K2) and the thiamine (vitamin B1) salvage pathway. Safety analysis was performed by searching for putative virulence factors using VirulenceFinder. The evaluation was performed by comparing the whole genome sequences of IBB3394 with known virulence genes of Enterococcus, E. coli, S. aureus and Listeria. The result indicates that there are no virulence genes in the IBB3394 genome. As a result, IBB3394 was found to have no toxic or pathogenic genes associated with the well-known pathogens Enterococcus, E. coli, S. aureus and Listeria. The result was also confirmed by whole-genome sequence analysis using another bacterial pathogenicity prediction web server, PathogenFinder. This tool did not classify strain IBB3394 as a pathogen. Genome analysis using various bioinformatics tools recommended by EFSA, such as ResFinder, CARD and RGI, and KEGG, did not reveal the presence of antibiotic resistance genes (ARGs) in the IBB3394 strain. Through sequencing analysis, the safety of using the strain as a probiotic and the strain for industrial, food and medical applications was confirmed.

### Example 13. Production of Sourdough Bread Containing Weissella cibaria IBB3394 (KKP 2094p)

Bread with the addition of bakery yeast was made using whole grain rye flour.

To produce the sourdoughs, the starter culture described in the patent PL238153B1 was used, the starter containing the strains Lactobacillus plantarum B/00117, Lactobacillus plantarum B/00118 and Lactobacillus brevis in a 1:1:1 ratio (Set 11) and a commercial starter culture LV2 [starter culture for acidifying rye flour (bacterial and yeast) by Lesaffre, containing Saccharomyces chevalieri, Lactobacillus brevis-data obtained from Lesaffre Bio-Corporation S.A., Łódź] as controls and the same strains as in the Set 11 supplemented with the strain W. cibaria IBB3394 (KKP 2094p) in a ratio of 1:1:1:1 as a test sample. Sourdoughs were prepared as in Example 3 described in the patent PL238153B1.

The bread (bakery product) was prepared according to the following recipe (for loaves of bread of 0.5 kg weight).

Preparation of raw materials and their quantities for approximately 1 kg of dough:

| | | |
|---|---|---|
| rye flour type 2000 | 0.5 | kg |
| sourdough from rye flour type 2000 | 0.17 | kg |
| bakery yeast | 0.006 | kg |
| salt | 0.012 | kg |

| | |
|---|---|
| water (temp. 38° C.) | approximately |
| | 0.31 kg |

Production and preparation of the proper I dough

| | | |
|---|---|---|
| kneading dough (dough up) - time - slow rotation | 7 | min. |
| kneading dough (dough up) time - fast rotation | 3 | min. |

| | |
|---|---|
| temperature of the proper dough | up to 35° C. |

Dividing, forming and shaping of the proper dough

| | | |
|---|---|---|
| weight of a piece | 0.5 | kg |
| time for dividing and shaping the dough into pieces | 45 | min. |

| | |
|---|---|
| fermentation time of the proper dough in the pieces | up to 90 min. |

Baking process

| | | |
|---|---|---|
| baking temperature: | 200° | C. |
| baking time | 60 | min. |

The methods described in PL238153B1 were used to evaluate bread. The obtained bread was organoleptically assessed according to PN-A-74108:1996 [the norm], the analysis was carried out using the point method by a panel with proven sensory sensitivity. After baking, the crumb moisture and crumb texture profile of the baked loaves of breads were analyzed. The microbiological stability of the produced bread was also determined. The obtained loaves of breads were stored for 7 days in order to determine the changes occurring during the aging of the bread. The individual results obtained for the tested bread produced using Set 11 with the addition of the strain W. cibaria IBB3394 (KKP 2094p) and two types of bread constituting controls, produced using Set 11 and produced using commercial culture (LV2), are presented in Tables 7-11.

For bread with the addition of sourdough made from W. cibaria IBB3394 (KKP 2094p), increased elasticity of the crumb was observed while maintaining the crispiness of the bread crust, as well as improvement in the taste and smell of the obtained bread compared to the control. Bread with the addition of W. cibaria IBB3394 (KKP 2094p) was characterized by a similar loaf volume and higher crumb moisture, significantly lower crumb hardness and chewiness compared to the control bread. Sourdough bread supplemented with W. cibaria IBB3394 (KKP 2094p) has the same microbiological stability (for 3 days) as the control bread. The resulting bread obtained a higher organoleptic rating than the control bread.

TABLE 7
Quality parameters of bread

| Type of | | | | | |
|---|---|---|---|---|---|
| culture | | | volume 100 g | bread | |
| used | weight | volume | of bread | efficiency | baking loss |
| LV2 | 410.4 ± 4.5a | 738.8 ± 18.4a | 180.1 ± 5.4a | 151.0 ± 1.7a | 17.92 ± 0.90a |
| Set 11 | 405.8 ± 2.4a | 748.0 ± 15.9a | 184.3 ± 3.4a | 149.3 ± 0.9a | 18.85 ± 0.47a |
| Set 11 + | 408.0 ± 3.6a | 743.7 ± 1.6a | 182.3 ± 1.2a | 150.1 ± 1.3a | 18.40 ± 0.72a |
| IBB3394 | | | | | |

TABLE 8
Organoleptic assessment

| | | | | other | | | | |
|---|---|---|---|---|---|---|---|---|
| Type of | | | | features | | | | |
| culture | | crust | crust | of the | crumb | crumb | other crumb | taste and |
| used | appearance | color | thickness | crust | elasticity | porosity | characteristics | smell |
| LV2 | 4.8 ± 0.4a | 3.0 ± 0.0a | 4.0 ± 0.0a | 4.0 ± 0.0a | 2.5 ± 1.2a | 2.8 ± 0.4a | 2.8 ± 0.4a | 5.2 ± 0.4a |
| Set 11 | 4.8 ± 0.4a | 3.0 ± 0.0a | 4.0 ± 0.0a | 4.0 ± 0.0a | 3.2 ± 0.4ab | 2.8 ± 0.4a | 2.8 ± 0.4a | 5.4 ± 0.5a |
| Set 11 + | 4.8 ± 0.4a | 3.0 ± 0.0a | 4.0 ± 0.0a | 4.0 ± 0.0a | 3.5 ± 0.7b | 2.8 ± 0.4a | 2.8 ± 0.4a | 5.5 ± 0.5a |
| IBB3394 | | | | | | | | |

TABLE 9
Crumb moisture

| Type of culture used | day 1 | day 2 | day 5 |
|---|---|---|---|
| LV2 | 49.39 ± 0.24b | 49.17 ± 0.24a | 48.41 ± 0.11b |
| Set 11 | 47.58 ± 0.34a | 48.29 ± 0.29a | 47.14 ± 0.08a |
| Set 11 + IBB3394 | 47.85 ± 0.34a | 48.25 ± 0.35a | 47.26 ± 0.30a |

TABLE 10
Changes in crumb hardness during storage

| Type of culture used | day 1 | day 2 | day 5 |
|---|---|---|---|
| LV2 | 28.0 ± 0.1c | 29.5 ± 2.1b | 36.1 ± 1.2c |
| Set 11 | 26.5 ± 1.0b | 24.1 ± 0.4a | 32.0 ± 0.4b |
| Set 11 + IBB3394 | 24.2 ± 0.6a | 21.7 ± 1.1a | 28.1 ± 1.7a |

TABLE 11
Changes in crumb chewiness during storage

| Type of culture used | day 1 | day 2 | day 5 |
|---|---|---|---|
| LV2 | 8.6 ± 0.4a | 10.7 ± 0.8b | 10.1 ± 2.0a |
| Set 11 | 9.4 ± 1.0a | 7.5 ± 0.2a | 9.0 ± 0.4a |
| Set 11 + IBB3394 | 8.1 ± 0.4a | 6.4 ± 0.6a | 7.9 ± 0.7a |

The bread prepared with the Set 11+IBB3394 culture was characterized by the lowest hardness of all the tested loaves of bread, regardless of the day of storage, what indicates a more delicate bread crumb, moreover the hardness in the case of bread produced with Set 11+IBB3394, increased by 16% compared to the 1st day, and in the case of the LV2 commercial culture by 28%, which indicates a much slower staling process, which could have been influenced by the exopolysaccharides produced during the fermentation of the sourdough.

### LITERATURE

[1] Aziz et al. (2008). The RAST Server: rapid annotations using subsystems technology. BMC genomics, 9 (1): 1-15.
[2] Badel et al. (2011). New perspectives for lactobacilli exopolysaccharides, Biotechnol Adv, 29:54-66.
[3] Baruah et al. (2016). Functional food applications of dextran from Weissella cibaria RBA12 from pummelo (Citrus maxima). Int J Food Microbiol, 242:124-31.
[4] Boyce and Havill (2005). Nosocomial antibiotic-associa-ted diarrhea associated with enterotoxin-producing strains of methicillin-resistant Staphylococcus aureus. Am J Gastroenterol, 100:1828-34.
[5] Boyce et al. (2007). Widespread environmental contamination associated with patients with diarrhea and methicillin-resistant Staphylococcus aureus colonization of the gastrointestinal tract. Infect Control Hosp Epidemiol, 28:1142-7.
[6] Buksa et al. (2021). Extraction, purification and characterisation of exopolysaccharides produced by newly isolated lactic acid bacteria strains and the examination of their influence on resistant starch formation. Food Chem, 362, 130221.
[7] Collins et al. (1993). Taxonomic studies on some Leuconostoc-like organisms from fermented sausages: description of a new genus Weissella for the Leuconostoc paramesenteroides group of species. J Appl Bacteriol, 75:595-603.
[8] Dey and Kang (2020). Weissella confusa DD_A7 pre-treatment to zebrafish larvae ameliorates the inflammation response against Escherichia coli 0157: H7. Microbiol Res, 237, 126489.
[9] EFSA (2018) Panel on Additives and Products or Substances used in Animal Feed (FEEDAP). Guidance on the characterisation of microorganisms used as feed additives or as production organisms. EFSA J 16 (3), e05206.
[10] EL-Adawi et al. (2012). Cytotoxicity assay and antioxidant activities of the lactic acid bacterial strains. African J Microbiol Res, 6 (8): 1700-12.
[11] Elshaghabee et al. (2020). Effect of oral administration of Weissella confusa on fecal and plasma ethanol concentrations, lipids and glucose metabolism in wistar rats fed high fructose and fat diet. Hepatic Med, 12:93-106.
[12] Han et al. (2016). Improvement of the texture of yogurt by use of exopolysaccharide producing lactic acid bacteria, BioMed Res Int, 7945675.
[13] Hu and Gänzle (2018) Effect of temperature on production of oligosaccharides and dextran by Weissella cibaria 10 M. Int J Food Microbiol 280:27-34.
[14] Jin et al. (2019). Isolation and characterization of high exopolysaccharide-producing Weissella confusa VP30 from young children's feces. Microbl Cell Factories, 18, 110.
[15] Kang et al. (2019). Safety evaluation of oral care probiotics Weissella cibaria CMU and CMS1 by phenotypic and genotypic analysis. Int J Mol Sci, 20 (11), 2693.
[16] Kanehisa et al. (2022). KEGG mapping tools for uncovering hidden features in biological data. Protein Science, 31 (1): 47-53.
[17] Kanehisa et al. (2016). BlastKOALA and GhostKOALA: KEGG tools for functional characterization of genome and metagenome sequences. J Mol Biol, 428 (4): 726-31.
[18] Kavitake et al. (2020). Overview of exopolysaccharides produced by Weissella genus—A review. Int J of Biol Macromol, 164:2964-73.
[19] Kavitake, et al. (2016). Characterization of a novel galactan produced by Weissella confusa KR780676 from an acidic fermented food. International journal of biological macromolecules, 86, 681-689.
[20] Kim et al. (2008). Characterization of exopolysaccharide (EPS) produced by Weissella hellenica SKkimchi3 isolated from kimchi. J Microbiol, 46 (5): 535-41.
[21] Lan et al. (2012). Bio-protective potential of lactic acid bacteria isolated from fermented wax gourd. Folia Microbiol, 57 (2): 99-105.
[22] Le et al. (2020). Synbiotic fermented soymilk with Weissella cibaria FB069 and xylooligosaccharides prevents proliferation in human colon cancer cells. J Appl Microbiol, 128 (5): 1486-96.
[23] Lee et al. (2012). Probiotic properties of Weissella strains isolated from human faeces. Anaerobe, 18:96-102.
[24] Lee et al. (2015). Multifunctional effect of probiotic Lactococcus lactis KC24 isolated from kimchi. LWT—Food Sci Technol, 64:1036-1041.
[25] Lim, et al. (2018). Characterization of antibacterial cell-free supernatant from oral care probiotic Weissella cibaria, CMU. Molecules, 23 (8), 1984.
[26] Litwinek et al. (2022). Developing lactic acid bacteria starter cultures for wholemealrye flour bread with improved functionality, nutritional value, taste, appearance and safety. PLOS ONE 17 (1), e0261677. [27] Lynch et al. (2018). Lactic acid bacteria exopolysaccharides in foods and beverages: isolation, properties, characterization, and health benefits, Annu Rev Food Sci Technol, 9:155-76.
[28] Mun and Chang (2020). Characterization of Weissella koreensis SK isolated from kimchi fermented at low temperature (around 0 degrees C.) based on complete genome sequence and corresponding phenotype. Microorganisms, 8, 1147.
[29] Ndagano et al. (2011). Antifungal activity of 2 lactic acid bacteria of the Weissella genus isolated from food. J Food Sci, 76 (6): M305-M311.
[30] Onanuga and Temedie (2011). Multidrug-resistant intestinal Staphylococcus aureus among self-medicated healthy adults in Amassoma, South-South, Nigeria. J Health Popul Nutr, 29 (5): 446-53.
[31] Patrone et al. (2021). Integrated phenotypic-genotypic analysis of candidate probiotic Weissella cibaria strains isolated from dairy cows in Kuwait. Probiotics Antimicrob Proteins, 13 (3), 809-23.
[32] Prado et al. (2020). Weissella paramesenteroides Wpk4 ameliorate the experimental amoebic colitis by increasing the expression of MUC-2 and the intestinal epithelial regeneration. J Appl Microbiol, 129 (6): 1706-19.
[33] Vosough et al. (2021). Evaluation of antioxidant, antibacterial and cytotoxicity activities of exopolysaccharide from Enterococcus strains isolated from traditional Iranian Kishk. Food Measure, 15:5221-30.
[34] Saadat et al. (2019). A comprehensive review of anticancer, immunomodulatory and health preferable effects of the lactic acid bacteria exopolysaccharides, Carbohydr Polym, 217:79-89.
[35] Sałański et al. (2022). Health-promoting nature of L. lactis IBB109 and IBB417 strains exhibiting proliferation inhibition and stimulation of interleukin-18 expression in colorectal cancer cells-insights at physiological and genomic level. Front Microbiol, 1825.
[36] Sandes et al. (2020). Weissella paramesenteroides WpK4 plays an immunobiotic role in gut-brain axis, reducing gut permeability, anxiety-like and depressive-like behaviors in murine models of colitis and chronic stress. Food Res Int, 137, 109741.
[37] Sokołowska-Wojdyło (2021). Infekcje gronkowcowe skóry—problem wciąż aktualny. Wiadomości dermatologiczne 11, Sep. 24, 2021
[38] Tao et al. (2017). Database and bioinformatics studies of probiotics. J Agric Food Chem, 65 (35): 7599-606.
[39] Teixeira et al. (2021). Weissella: an emerging bacterium with promising health benefits. Probiotics Antimicrob Proteins, 13:915-25.
[40] Torino et al. (2015). Biopolymers from lactic acid bacteria. Novel applications in foods and beverages. [Review]. Front Microbiol, 6, 834.
[41] Valerio et al. (2020). Effect of Amaranth and Quinoa Flours on exopolysaccharide production and protein profile of liquid sourdough fermented by Weissella cibaria and Lactobacillus plantarum. Front Microbiol 11, 967.
[42] Vasanthakumari et al. (2015). Physicochemical characterization of an exopolysaccharide produced by a newly isolated Weissella cibaria. Appl Biochem Biotechnol, 176 (2): 440-53.
[43] Wolter et al. (2014) Evaluation of exopolysaccharide producing Weissella cibaria MG1 strain for the production of sourdough from various flours. Food Microbiol 37:44-50.
[44] Xu et al. (2017). In situ synthesis of exopolysaccharides by Leuconostoc spp, and Weissella spp, and their rheological impacts in fava bean flour. Int J Food Microbiol, 248:63-71.
[45] Yeu et al. (2021). Antimicrobial and antibiofilm activities of Weissella cibaria against pathogens of upper respiratory tract infections. Microorganisms, 9 (6), 1181.
[46] Yildiz and Karatas (2018). Microbial exopolysaccharides: Resources and bioactive properties. Process Biochemistry, 72:41-6.
[47] Yu et al. (2019a). Antagonistic and antioxidant effect of probiotic Weissella cibaria JW15. Food Sci Biotechnol, 28:851-5.
[48] Yu et al. (2019b). Anti-Inflammatory potential of probiotic strain Weissella cibaria JW15 isolated from kimchi through regulation of NF-κB and MAPKs pathways in LPS-induced RAW 264.7 cells. J Microbiol Biotechnol, 29:1022-32.
[49] Zeidan et al. (2017). Polysaccharide production by lactic acid bacteria: From genes to industrial applications. FEMS Microbiol Rev, 41 (Supp_1): S168-S200.
[50] Zhang et al. (2018) Characterization of a yogurt-quality improving exopolysaccharide from Streptococcus thermophilus AR333, Food Hydrocoll, 81:220-8.
[51] Zhou et al. (2018) Exopolysaccharides of lactic acid bacteria: structure, bioactivity and associations: a review, Carbohydr Polym, 207:317-32.

1. A new bacterial strain Weissella cibaria, deposited in the IAFB (IAFB Collection of Industrial Microorganisms of prof. Waclaw Dabrowski, Warsaw, Poland) under the deposit no. KKP 2094p.

2. A composition comprising the new bacterial strain Weissella cibaria according to claim 1, deposited in the IAFB under the deposit no. KKP 2094p.

3. The composition according to claim 2, wherein it is a pharmaceutical composition further comprising a pharmaceutically acceptable carrier, wherein preferably the composition is in the form of a liquid, solid, powder, tablet, capsule intended for oral administration.

4. A pharmaceutical composition comprising the bacterial strain Weissella cibaria as defined in claim 1 and a pharmaceutically acceptable carrier for use as a medicament.

5. The pharmaceutical composition for use according to claim 4, wherein it is for use as a medicament for the prevention and/or treatment of intestinal cancer, preferably for the prevention and/or treatment of colorectal cancer, most preferably colorectal adenocarcinoma.

6. A dietary supplement comprising the bacterial strain Weissella cibaria as defined in claim 1, wherein the dietary supplement is preferably in the form of a liquid, solid, powder, tablet, capsule intended for oral administration.

7. A probiotic preparation comprising the bacterial strain Weissella cibaria as defined in claim 1, wherein the probiotic preparation preferably further comprises prebiotic substances, preferably selected from oligosaccharides, polysaccharides, fructo-oligosaccharides, lactulose, inulin, resistant starch, cellulose, hemicellulose and pectins, wherein the preparation preferably also comprises postbiotics, wherein preferably the postbiotics are selected from short-chain fatty acids, enzymes, lipopolysaccharides, teichoic acids, vitamins, butyric acid, acetate, propionate, muramyl dipeptide, indole, teichoic acid, lactocepins.

8. The probiotic preparation according to claim 7, wherein it is in the form of a liquid, solid, powder, tablet, capsule intended for oral administration.

9. A bacterial starter culture for making bread, comprising the strain Weissella cibaria as defined in claim 1.

10. A sourdough for making bread, comprising the bacterial starter culture as defined in claim 9.

11. A method for the production of bread, comprising a step of adding the bacterial starter culture as defined in claim 9.

12. A bread, comprising the bacterial starter culture as defined in claim 9.

13. A method for the microbiological production of dextran, comprising a step in which the bacterial strain of Weissella cibaria as defined in claim 1 is cultured in a liquid medium without dextrose (without glucose) with an addition of saccharose, an organic nitrogen source containing amino acids and short peptides, B vitamins and mineral salts.

14. The method according to claim 13, wherein the culture is carried out on MRS medium (DeMan-Rogosa-Sharpe broth), a medium for lactic acid bacteria, preferably with the addition of 5-10%±2 by wt. of saccharose.

15. A bacterial preparation comprising the bacterial strain Weissella cibaria as defined in claim 1 for use as an active ingredient in a probiotic, a therapeutic preparation, a functional food, a dietary supplement, for use as an active ingredient in a drug intended for prevention and/or treatment of intestinal cancer, preferably colon cancer.

16. Use of the bacterial strain Weissella cibaria as defined in claim 1 as a functional additive in a functional food, a functional additive in a food drink, as an active ingredient of a probiotic preparation, a bacterial preparation, a dietary supplement, a pharmaceutical composition.

17. Use of the bacterial strain Weissella cibaria as defined in claim 1 in the food industry as an emulsifier, thickener, stabilizer or texture improver of a food product.

18. Use of the bacterial strain Weissella cibaria as defined in claim 1 for the microbiological production of exopolysaccharides (EPS), preferably dextran.

19. Use of the bacterial strain Weissella cibaria as defined in claim 1 for the production of sourdough for bread.

20. Use of the bacterial strain Weissella cibaria as defined in claim 1 for making bread.
